(19) United States
(12) Patent Application Publication
(10) Pub. No.: US 2013/0023042 A1
(43) Pub. Date: Jan. 24, 2013

(54) METAL BUFFER LAYER ASSISTED GUIDED MODE RESONANCE BIOSENSOR

(71) Applicants: Jenq-Yang CHANG, TAOYUAN COUNTY (TW); Wen-Yih CHEN, TAOYUAN COUNTY (TW); Chih-Cheng CHIEN, TAOYUAN COUNTY (TW); Sheng-Fu LIN, TAOYUAN COUNTY (TW)

(72) Inventors: Jenq-Yang CHANG, TAOYUAN COUNTY (TW); Wen-Yih CHEN, TAOYUAN COUNTY (TW); Chih-Cheng CHIEN, TAOYUAN COUNTY (TW); Sheng-Fu LIN, TAOYUAN COUNTY (TW)

(21) Appl. No.: 13/323,284

(22) Filed: Dec. 12, 2011

(30) Foreign Application Priority Data
Jul. 19, 2011 (TW) 100125528

(51) Int. Cl. C12M 1/40; G01N 30/96

(52) U.S. Cl. 435/288.7; 422/69

(57) ABSTRACT

A metal buffer layer assisted guided mode resonance (GMR) biosensor is disclosed. The GMR biosensor includes a substrate, a metal buffer layer and a waveguide layer. The metal buffer layer is disposed on the substrate and the waveguide layer is disposed on the metal buffer layer. The metal buffer layer, which is disposed adjacent to the waveguide layer, can carry out the total reflection and provide extra phase compensation of the total reflection at the same time. Accordingly, the propagation constant of the resonance wave would be much closer to the sensitivity of the phase, and the resonance electric field of the GMR biosensor would be much closer to the sensitive area. Consequently, the sensitivity of the GMR biosensor could be improved.

### CROSS REFERENCE TO RELATED APPLICATIONS

This Non-provisional application claims priority under 35 U.S.C. §119(a) on Patent Application No(s). 100125528 filed in Taiwan, Republic of China on Jul. 19, 2011, the entire contents of which are hereby incorporated by reference.

### BACKGROUND OF THE INVENTION

1. Field of Invention

The present invention relates to a biosensor and, in particular, to a guided mode resonance (GMR) or guided wave resonance or resonant waveguide or photonic crystal biosensor.

2. Related Art

Various kinds of sensors are introduced in different applications. For example, many electronic products, vehicles and machines must be equipped with a sensor. Some sensors usually detect the changes of physical properties such as temperature, light and pressure. These sensors are called physical sensors such as thermometers or pressure gauge. Besides, some sensors are mainly applied to detect the chemical materials, so they are also called chemical sensors such as a pH electrode (pH meter).

However, the conventional chemical sensors can only detect the inorganic materials rather than the organic materials. In fact, the detection of the bio organic materials in the fields of bio-industrials, clinical diagnosis, and environmental engineering is very important. The conventional detecting methods for bio organic materials include the chromogenic method, piezoelectric method, and optical spectrum analysis method. However, these methods have the disadvantages of time consuming and expensive equipments. Consequently, it is desired to develop a new biosensor for detecting organic materials.

For developing a biosensor with a specific function, it is important to consider the characteristics of the selected bio molecules, mechanisms, signal generating/outputting modes, concentrations, operation environment parameters. The conventional biosensors include (1) electrochemical biosensors, (2) semiconductor transistor sensors, (3) optical biosensors, and (4) piezoelectric quartz crystal biosensors. Regarding to the optical biosensors, there are several popular and critical technologies including the evanescence wave technology, surface plasma resonance technology, and fluorescent labeling technology. The fluorescent labeling technology includes a step of labeling the target, so the operation time increases and the chemical reaction is more complicated. The surface plasma resonance technology does not need the labeling step, but it needs more detecting space for improving its sensitivity and stability, which restricts the minimum size of the system design with using this technology. Besides, the surface plasma resonance technology is hard to achieve the high-throughput requirement.

The guided mode resonance (GMR) technology for biosensing does not need the florescent labeling step, can carry out the fast sampling process, can be manufactured in mass production, can have high capacity, and can be manufactured by semiconductor processes so as to achieve the minimized size, combine with other semiconductor devices, and have no limitation in the size of the target to be detected. Therefore, it has become one of the most popular biosensors. The GMR biosensor mainly includes a grating element on or in a planer waveguide element. By changing the surface effective refractive indexes, the boundary conditions on the wave guide are changed. Similarly, the guided-wave in the wave guide may have the optical properties with wavelength shift due to the changed boundary conditions.

Generally, the grating element can provide the momentum in the direction parallel to the interface to the incident light field within the spatial frequency domain. When the wavelength of the incident light is a resonance wavelength of the grating element, which means the wavelength of the incident light matches the phase matching and the coupling condition, the total internal reflection occurs in the boundary area of the wave guide and coupled to the wave guide. In the wave guide, the diffraction light can generate the total internal reflection at the interface of the wave guide and the substrate, and then couple out to the air through the upper grating interface. The light outputted through the grating can interfere with the incident light to form the reflective light. The zero-order light can perpendicularly pass through the wave guide (this is the detectable transmission spectrum). The phase matching and the coupling conditions of the resonance wavelength are very sensitive with the environmental optical refraction index, so it is suitable for configuring a biosensor.

Therefore, it is desired to develop a GMR biosensor with high sensitivity.

### SUMMARY OF THE INVENTION

In view of the foregoing, an objective of the present invention is to provide a GMR biosensor with high sensitivity.

To achieve the above objective, the present invention discloses a guided mode resonance (GMR) biosensor including a substrate, a metal buffer layer and a waveguide layer. The metal buffer layer is disposed on the substrate, and the waveguide layer is disposed on the metal buffer layer.

In one embodiment, the waveguide layer includes a grating structure.

In one embodiment, the GMR biosensor further includes a grating layer disposed on the waveguide layer.

In one embodiment, the material of the waveguide layer includes photonic crystals.

In one embodiment, the substrate includes a plurality of microstructures, and the metal buffer layer and the waveguide layer are disposed on the microstructures in order.

In one embodiment, the substrate is opaque or light-permeable.

In one embodiment, the thickness of the waveguide layer is between 50 nm and 1000 nm.

In one embodiment, the thickness of the metal buffer layer is larger than 50 nm.

In one embodiment, the reflectivity of the metal buffer layer is substantially larger than 90%.

In one embodiment, the thickness of the grating structure or grating layer is less than 1 μm.

As mentioned above, the GMR biosensor of the present invention has a metal buffer layer for carrying out the total reflection of the signals, so that the second total reflection angle is unnecessary so as to enhance the displacement of the resonance wavelength caused by the bio molecules. Besides, since the metal buffer layer can provide extra phase compensation of total reflection, the energy of the optical field may be close to the surface of the GMR biosensor. This can further improve the signal sensitivity.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a schematic diagram showing a GMR biosensor with a metal buffer layer according to a preferred embodiment of the invention;

FIG. 1B is a schematic diagram showing another GMR biosensor with a metal buffer layer according to the preferred embodiment of the invention;

FIG. 1C is a schematic diagram showing another GMR biosensor with a metal buffer layer according to the preferred embodiment of the invention;

FIG. 1D is a schematic diagram showing another GMR biosensor with a metal buffer layer according to the preferred embodiment of the invention;

FIGS. 2A and 2B are schematic diagrams showing the GMR biosensor with a metal buffer layer of the invention which is operated in the detecting procedures;

FIG. 3A is a phase diagram of the GMR biosensor with a metal buffer layer of the invention;

FIG. 3B is a phase diagram of a conventional GMR device; and

FIGS. 4A and 4B are schematic diagrams showing the energy intensity of the optical fields of the GMR biosensor with a metal buffer layer of the invention and the conventional GMR device.

### DETAILED DESCRIPTION OF THE INVENTION

The present invention will be apparent from the following detailed description, which proceeds with reference to the accompanying drawings, wherein the same references relate to the same elements.

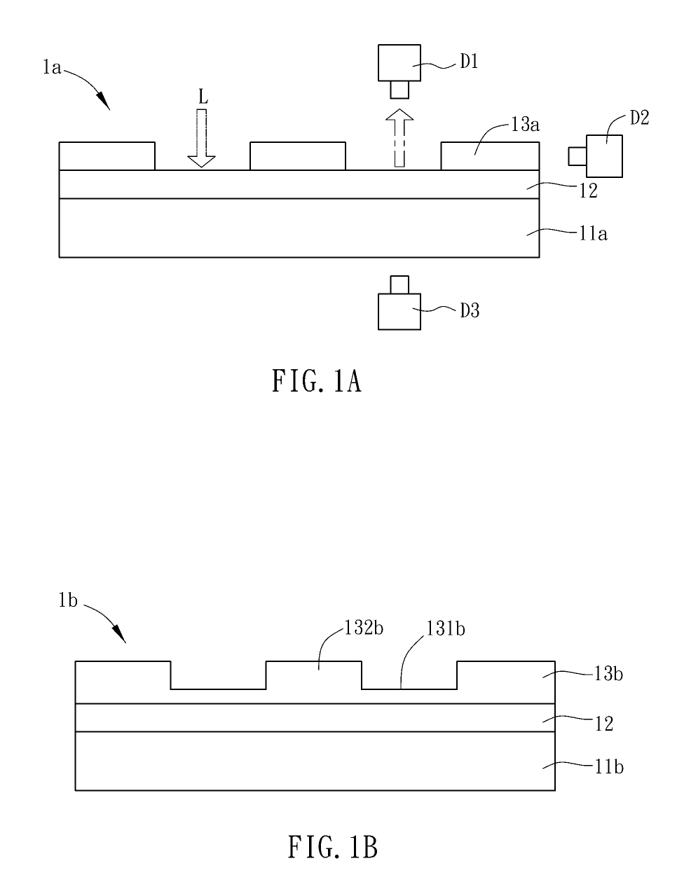

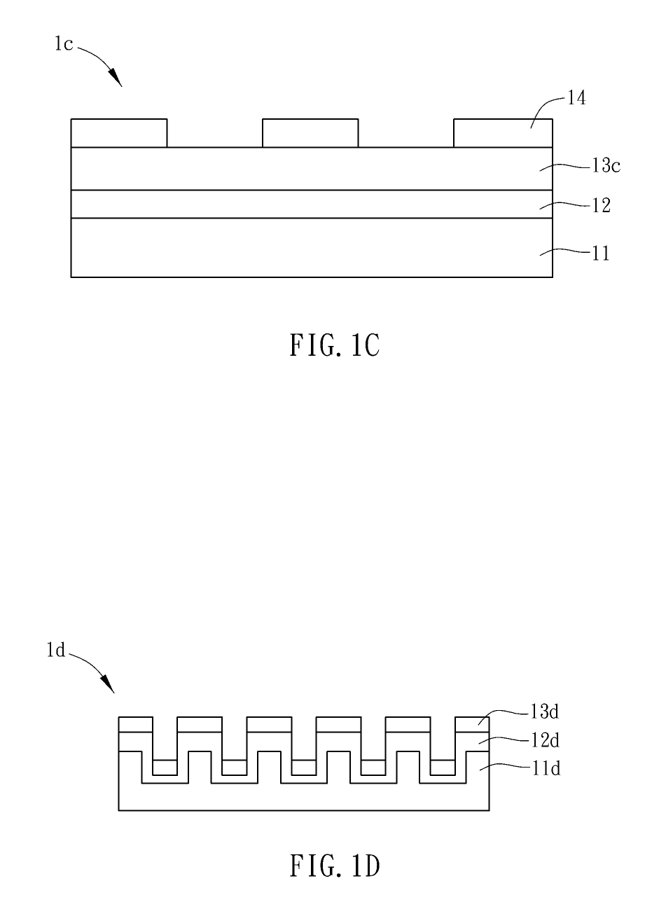

With reference to FIG. 1A, a guided mode resonance (GMR) biosensor 1a according to a preferred embodiment of the invention includes a substrate 11a, a metal buffer layer 12 and a waveguide grating layer 13a. The metal buffer layer 12 is disposed on the substrate 11a, and the waveguide layer 13a is disposed on the metal buffer layer 12. The GMR biosensor 1a can be used to detect the concentration or any property of bio or chemical material with binding specificity. For example, the bio or chemical material can be a DNA, RNA, nucleotide, peptide, protein, enzyme, antibody, antigen, etc.

If the substrate 11a is a light-permeable substrate, such as a glass substrate or quartz or transparent plastic substrate, the incident light L emitted from the light source enters the GMR biosensor 1a through the waveguide layer 13a. The signal sensor D1, D3 and D2 are disposed at a light input side of the substrate 11a of the GMR biosensor 1a, the opposite side, and a side parallel to the waveguide layer 13a, respectively, for receiving the light refracted and reflected by the waveguide layer 13a, the light passing through the waveguide layer 13a, and the lateral leaked light. The detected result can be further calculated and analyzed. If the substrate 11a is an opaque substrate, such as a ceramic substrate, a printed circuit substrate, or a metal substrate, the signal sensors D1 and D2 can still receive the light signals. The incident light L from the light source can enter the GMR biosensor 1a after passing through a fiber, or the light source can be a general collimation light source. Besides, the incident light L can be a polarized light or non-polarized light. In this embodiment, the incident light L is a polarized light for example. In this case, a polarizer is configured between the fiber and the GMR biosensor 1a for modulating the incident light L into a polarized light.

The thickness of the metal buffer layer 12 is larger than 50 nm. The material of the metal buffer layer 12 may include gold, aluminum, silver or platinum. The reflectivity of the metal buffer layer is substantially larger than 90%. In practice, the metal buffer layer 12 of the embodiment is not a light-permeable layer.

In this embodiment, the waveguide layer 13a includes a grating structure. The thickness of the waveguide layer 13a is between 50 nm and 1000 nm, and the material thereof includes photonic crystals. The grating period is smaller than the resonance wavelength which is included in the incident light L. Since the grating structure of the waveguide layer 13a has a certain thickness, it can used as a waveguide layer, which has both the grating and wave guiding functions. Of course, the waveguide layer and the grating structure may have different aspects. For example, as shown in FIG. 1B, the waveguide layer 13b has a planar portion 131b, and a grating structure 132b. Alternatively, as shown in FIG. 1C, the GMR biosensor 1c further includes a grating layer 14 disposed on the waveguide layer 13c. The thickness of either the grating structure or the grating layer 14 is smaller than 1 μm. In addition, the grating structure may be formed by the microstructures of the substrate. As shown in FIG. 1D, the substrate 11d has a plurality of microstructures having the shape similar to the grating structure. Then, the metal buffer layer 12d and the waveguide layer 13d are formed thereon in order by sputtering or depositing.

Referring to FIG. 1A again, the incident light L passes through the grating and then coupled to the waveguide layer 13a, thereby forming the resonance transmission, which called guide-mode resonance. In this embodiment, the waveguide layer 13a may include a grating layer. After passing through the grating layer, the incident light L may match the couple state of the wave guide under a specific bandwidth and incident angle. In more details, the incident light L with this specific bandwidth can enter the waveguide layer 13a and be transmitted in resonance therein. During performing the transmission spectrum scan of the incident light L, the transmission of the incident light may dramatically descend at a specific wavelength, which can indicate a detecting signal.

The light may leave the waveguide layer 13a through two interfaces. The first interface is between the waveguide layer 13a and the buffer solution of the sample (the GMR biosensor 1a is immersed in water or other buffer solution), and the second interface is between the waveguide layer 13a and the metal buffer layer 12. In the prior art, the second interface is between the waveguide layer and the transparent substrate. The critical angle of the second interface is larger than that of the first interface. If the propagation angle of the resonance light inside the waveguide layer 13a with respect to the interface is smaller than any critical angle of the two interfaces, the guide-mode resonance as well as the measurement of the signal fails. When the bio molecules are suspended or dissolved in water and bind to the surface of the grating structure through the immobilized ligand molecules, the detected resonance wavelength signal may be shifted toward the longer wavelength. Moreover, if the concentration of the bio molecules increases, the offset of the resonance wavelength increases too. The present invention configures the metal buffer layer 12 between the substrate 11a and the waveguide layer 13a so as to create the total reflection, so that it is easier to reach the first critical angle (smaller critical angle). Therefore, the resonance condition may approach to the high sensitivity conditions without being interfered by the second critical angle. Moreover, the present invention can increase the offset of the resonance wavelength and enhance the sensitivity for detecting the bio molecules. An application of the GMR biosensor of the present invention will be described hereinafter. In the following example, the GMR biosensor 1c as shown in FIG. 1C is adopted.

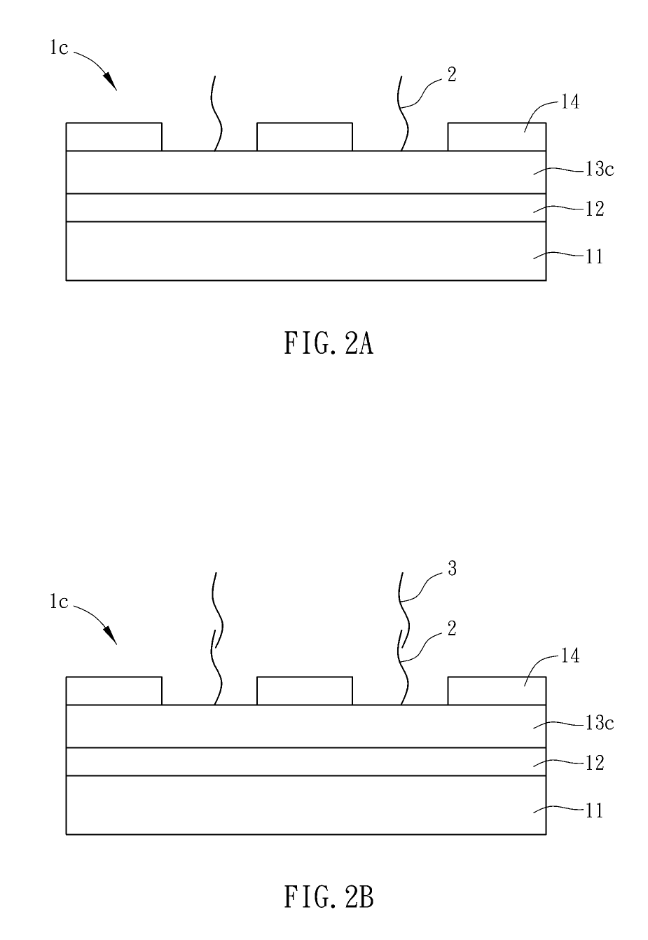

FIGS. 2A and 2B are schematic diagrams showing the GMR biosensor with a metal buffer layer of the invention which is operated in the detecting procedures.

A receptor 2 (e.g. an antibody or a single-strand DNA sequence) is fixed to the surface of the GMR biosensor 1c. In more detailed, the receptor 2 is bound to the surface of grating layer 14. The GMR biosensor 1c containing the fixed receptor 2 is then immersed in the liquid specimen. The liquid specimen must contact with the grating layer 14 inside the GMR biosensor 1c. The liquid specimen contains the ligand 3 (target) for conjugating with the receptor 2. For example, the ligand 3 can be a corresponding antigen or another single-strand DNA sequence. After a proper interaction period, the receptor 2 and the ligand 3 can spontaneously bind with each other by attaching or bond based on their specificity. As shown in FIG. 2B, the ligand 3 is bound with the receptor 2 fixed on the GMR biosensor 1c. Then, the GMR biosensor 1c with the receptor 2 and ligand 3 is detected.

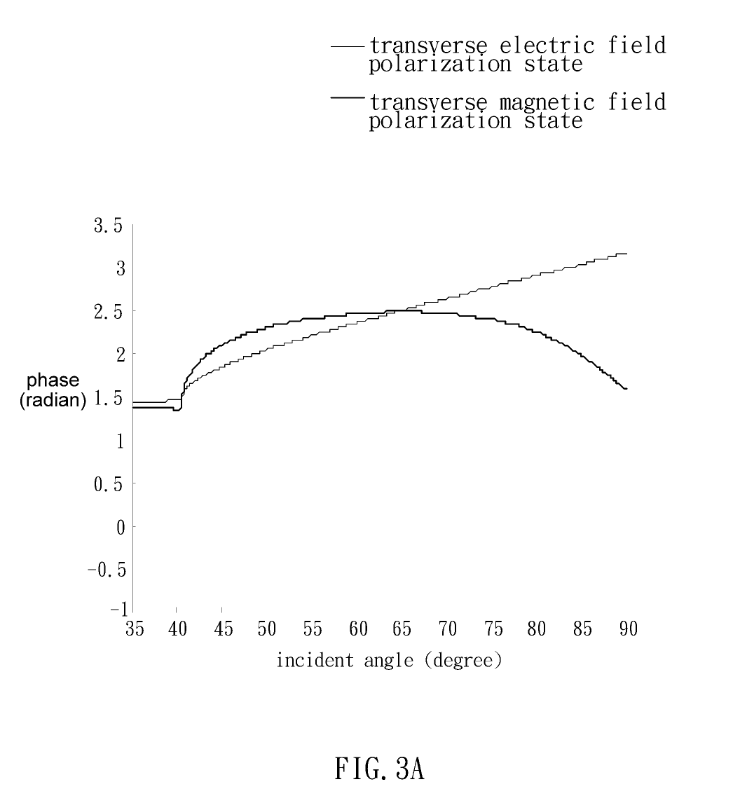

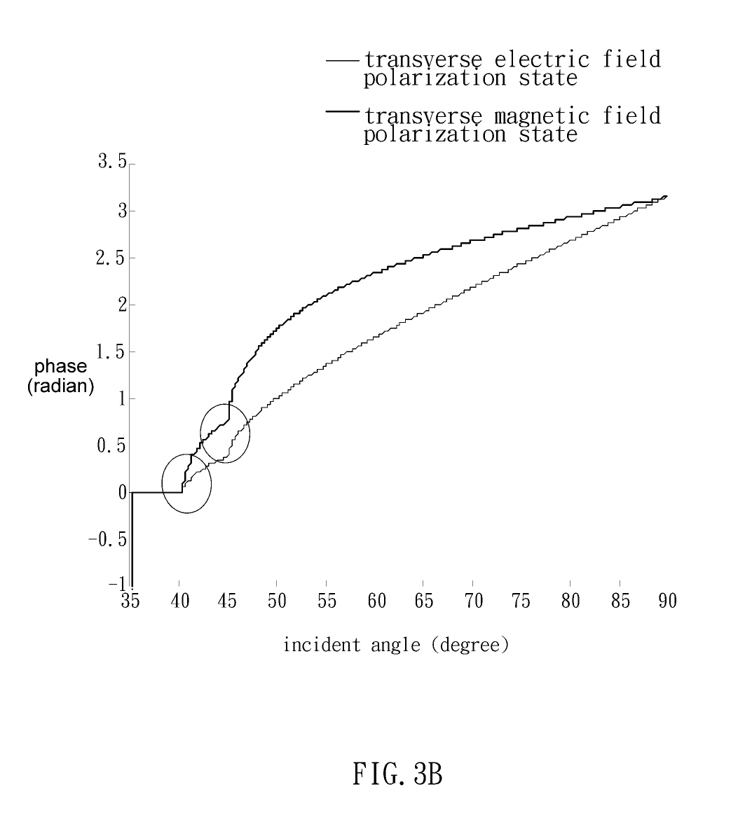

FIG. 3A is a phase diagram of the GMR biosensor with a metal buffer layer of the invention, and FIG. 3B is a phase diagram of a conventional GMR device, which does not configured with a metal buffer layer. FIG. 3B shows two obvious sharp turning points, which indicate two critical angles, while FIG. 3A shows only one sharp turning point in both the TE and TM modes, which indicate one critical angle. The reflectivity of the metal buffer layer is substantially larger than 90%. In other words, the metal buffer layer has a planar structure with high reflectivity.

Accordingly, although there are two interfaces between the liquid specimen, the waveguide layer and the metal buffer layer, the interface between the waveguide layer and the metal buffer layer can generate total reflection for the light beams from any angle. Thus, the incident light traveling to the metal buffer layer can have total internal reflection without considering the relation between the incident angle and the critical angle. Compared with the conventional GMR device, the GMR biosensor of the present invention has a metal buffer layer disposed at one side of the waveguide layer, so the critical angle between the conventional waveguide layer and substrate does not exist. Therefore, only one critical angle between the liquid specimen and the waveguide layer remains. In the GMR biosensor of the present invention, the incident angle for causing resonance has broader limit, which is to be larger than the critical angle of the interface between the liquid and the waveguide layer.

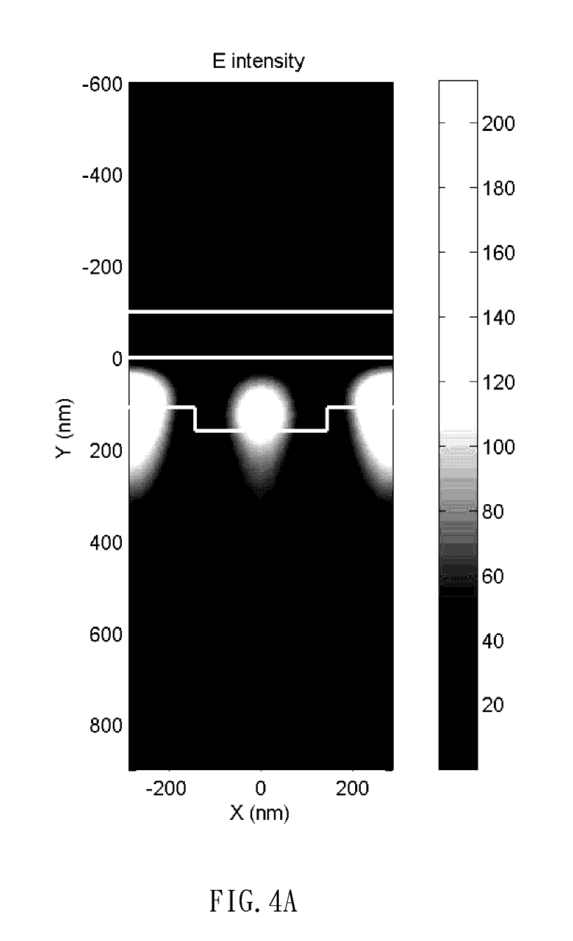

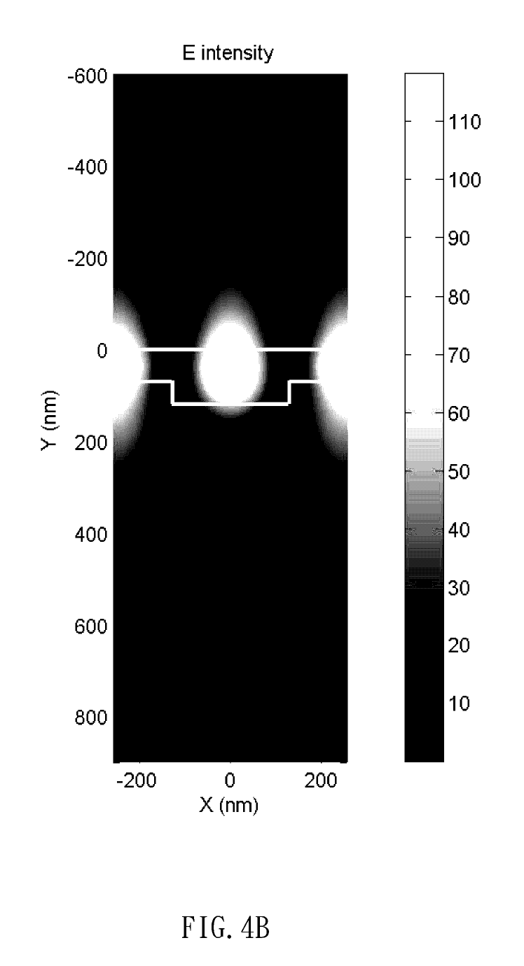

FIGS. 4A and 4B are schematic diagrams showing the energy intensity of evanescence wave of the GMR biosensor with a metal buffer layer of the invention and the conventional GMR device. FIG. 4A shows, form top to bottom, a glass substrate, a metal buffer layer, a waveguide layer, a grating layer and water, and FIG. 4B shows, form top to bottom, a glass substrate, a waveguide layer and water. In the energy distribution figure, the brighter portion (white) represents the stronger intensity area in the optical field. Referring to FIGS. 4A and 4B, the GMR biosensor with a metal buffer layer of the invention can provide extra total reflection phase compensation and can prevent the optical field from penetrating to the substrate. Thus, the energy of the optical field can not apply to the substrate and can be restricted around the waveguide layer. Besides, the energy distribution is asymmetric, so the overlap portion of the energy and the bio molecules located on the surface of the waveguide layer is increased. Herein, the overlap portion represents the high energy region. In the conventional GMR device, the strongest intensity area of the optical energy is located around the waveguide layer too, but some energy of the optical field penetrates to the substrate. This can cause the undesired energy dissipation. It is obvious that the energy applied to the bio molecules on the surface of the waveguide layer in the conventional GMR device is less than that in the GMR biosensor of the present invention. The above phenomenon can also support the conclusion that the GMR biosensor of the present invention can provide enhanced detection sensitivity.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A guided mode resonance (GMR) biosensor, comprising:
a substrate;
a metal buffer layer disposed on the substrate; and
a waveguide layer disposed on the metal buffer layer.

2. The GMR biosensor of claim 1, wherein the waveguide layer comprises a grating structure.

3. The GMR biosensor of claim 1, further comprising:
a grating layer disposed on the waveguide layer.

4. The GMR biosensor of claim 1, wherein the material of the waveguide layer comprises photonic crystals.

5. The GMR biosensor of claim 1, wherein the substrate comprises a plurality of microstructures, and the metal buffer layer and the waveguide layer are disposed on the microstructures in order.

6. The GMR biosensor of claim 1, wherein the substrate is opaque or light-permeable.

7. The GMR biosensor of claim 1, wherein the thickness of the waveguide layer is between 50 nm and 1000 nm.

8. The GMR biosensor of claim 1, wherein the thickness of the metal buffer layer is larger than 50 nm.

9. The GMR biosensor of claim 1, wherein the material of the metal buffer layer comprises gold, aluminum, silver or platinum.

10. The GMR biosensor of claim 1, wherein the reflectivity of the metal buffer layer is substantially larger than 90%.

11. The GMR biosensor of claim 2, wherein the thickness of the grating structure is less than 1 μm.

12. The GMR biosensor of claim 3, wherein the thickness of the grating layer is less than 1 μm.
